(19) United States
(12) Patent Application Publication
(10) Pub. No.: US 2014/0212826 A1
(43) Pub. Date: Jul. 31, 2014

(54) MEASUREMENT APPARATUS, METHOD FOR INVESTIGATING COATINGS ON A COATING PROBE, INCINERATION PLANT AND METHOD FOR OPERATING SUCH AN INCINERATION PLANT

(71) Applicant: MARTIN GMBH FUER UMWELT- UND ENERGIETECHNIK, Muenchen (DE)

(72) Inventors: Robert VON RAVEN, Seeshaupt (DE); Michael BECKMANN, Dresden (DE); Slawomir ROSTOWSKI, Dresden (DE); Wolfgang SPIEGEL, Mering (DE)

(73) Assignee: MARTIN GMBH FUER UMWELT- UND ENERGIETECHNIK, Muenchen (DE)

(21) Appl. No.: 14/163,013

(22) Filed: Jan. 24, 2014

(30) Foreign Application Priority Data
Jan. 28, 2013 (DE) 10 2013 001 361.5
Jul. 4, 2013 (DE) 10 2013 011 149.8

(51) Int. Cl. G01N 33/32; F27D 25/00; F22B 37/48; F27D 21/04

(52) U.S. Cl. CPC G01N 33/32; F27D 21/04; F27D 25/008; F22B 37/48
USPC 432/2; 73/150 R; 432/32

(57) ABSTRACT

The invention relates to a measuring apparatus comprising a coating probe and a probe holder which is disposed on a wall of a sample chamber. The coating probe is displaceable into the sample chamber on one side and is displaceable out from the sample chamber on the opposite side. As a result, practical investigations of boiler coatings are made possible.

The invention relates to a measurement apparatus, a method for investigating coatings on a coating probe, an incineration plant through which combustion gases can flow and a method for operating such an incineration plant.

In particular, the invention relates to a measurement apparatus comprising a coating probe and a probe holder and a method for investigating coatings on a coating probe in which the coating probe is disposed in an incineration plant.

In incineration plants particularly when using solid fuels, coatings form on the heat transfer surfaces. These coatings hinder the heat transport from the flue gas of the solid fuel to the working medium. The substances contained in the coatings can however also promote corrosion processes at the surface of the boiler wall or at heat exchanger tubes. Ultimately as a result of the insulating effect of the coatings on the radiating flues of larger incineration plants, this results in a shift of the temperature profile along the flue gas path so that downstream heat transfer surfaces are exposed to an increased thermal loading.

In order to avoid these negative effects of coating formation, the coatings are removed with the aid of cleaning systems, preferably during operation of the firing system.

In order to check the effect of a cleaning system of these coating layers, a measurement apparatus with a coating probe and a probe holder is used. The probe holder enables a probe to be positioned in a firing system. To this end the coating probe is inserted through an opening in the boiler wall into the flue gas region and held by means of the probe holder on the boiler wall. After the coating probe has been exposed to the flue gases for a defined time, it can be removed from the flue gas path so that the coatings adhering to the probe can be investigated. Such coating investigations are appropriate in order to determine the time of cleaning and specify the type of cleaning method. For example, water droplets can be sprayed onto the coatings via nozzles, which should vaporise on the coatings in such a way that they bring about a flaking of the coating.

However, it has been found that a cleaning method carried out in the laboratory to remove coatings on the coating probe does not necessarily also lead to correspondingly positive results during the removal of coatings in practice.

It is therefore the object of the invention to further develop a generic measuring apparatus comprising a coating probe and a probe holder to provide an improved method for investigating coatings on a coating probe, an incineration plant and a method for operating such an incinerating plant.

This object is solved in terms of apparatus by a generic measuring apparatus in which the probe holder is disposed at a wall of a sample chamber and the coating probe is displaceable on one side into the sample chamber and is displaceable on an opposite side out from the sample chamber.

Such a sample chamber can be disposed on the wall of a firing system and allows the coating probe to be withdrawn from the firing chamber into the sample chamber and investigated in the sample chamber. The sample chamber enables the atmospheric conditions such as in particular the temperature to be matched to the temperature in the firing chamber and to conduct the investigations under ambient conditions similar to the firing chamber without the coating probe being exposed to colder gases and in particular air humidity after removal from the firing chamber.

In order to achieve conditions similar to the firing chamber in the sample chamber, flue gases can flow from the firing chamber through the sample chamber. In order to avoid any cooling of the gases in the sample chamber, it is proposed that the sample chamber is heated.

In addition, the cooling can be reduced by thermally insulating the sample chamber.

In the sample chamber the coating probe can be exposed to different measuring devices. At the same time, the strength of the coating can be tested mechanically. The composition of the coatings can be determined by chemical methods and ultrasound and radiation methods can provide information on the type of coatings. Since the measuring devices required for this should not be exposed to the atmospheric conditions prevailing in the sample chamber, it is proposed that the sample chamber has a measuring window.

For simple visual investigations it is proposed that the sample chamber has a viewing window.

In order to investigate how a coating reacts to impinging water droplets, it is proposed that the sample chamber has a dropper. This dropper can make individual drops of different size fall onto the probe. However it can also have a spraying device such as a nozzle by which means water droplets or chemicals are applied to the coating probe.

An advantageous embodiment provides that the sample chamber has two opposite sealable coating probe passages. This enables the usually long probe to be drawn through the sample chamber and after removal of the probe from the boiler chamber close the coating probe passage pointing towards the boiler chamber. The sample chamber can thereby be separated from the firing chamber whilst the coating probe is located in the sample chamber.

This enables the sample chamber to be investigated in a laboratory and to carry out the measurement far removed from the firing system. The heating of the sample chamber then serves to maintain firing-chamber-like conditions during transport and the investigations so that the coating on the measuring probe is not changed by the atmosphere surrounding it.

In order to create realistic conditions on the coating probe, it is proposed that the coating probe has fluid lines for influencing the probe temperature. The coating probe can thereby be temperature-controlled by means of hot air, water, oil etc. in such a manner that the surface temperature on the coating probe corresponds to the surface temperatures at the heat transfer surfaces in the boiler. The fluid lines however also enable the setting of a temperature gradient at the surface of the probe in order to investigate the deposition and the coating qualities on the probe as a function of the surface temperature of the probe.

In particular with different surface temperatures on the probe it is proposed that the coating probe has temperature measuring devices. This enables the probe temperature to be set, to be regulated and to be varied arbitrarily.

The temperature monitoring and in particular a temperature regulation has the result that the coating is deposited at a known surface temperature of the substrate. To this end a specific surface temperature can be set by means of a cooling of the coating probe. In addition it is advantageous if the temperature of the sample chamber with the dropper is measured and preferably set or even regulated to determine the influence of the online cleaning on the pipes and their cooling.

The object forming the basis of the invention is also solved by a generic method in which the coating probe is drawn from the incineration plant directly into a sample chamber heated to above 100° C. which adjoins the incineration plant and is investigated there.

The measuring apparatus and in particular the method according to the invention thus enable coating probes to be investigated in temperature and gas environments which are not usually present during the investigation of a coating probe. Specifically the transfer of the coating probe from the incineration plant directly into the sample chamber in which the investigations are then conducted, yields investigation results which can be transferred to the situation of the coatings formed in the boiler. The method thus enables repeated measurements to be made on a coating probe without the coating probe cooling down or absorbing moisture. These measurement results can be used to determine how and when coatings formed in the boiler can be effectively removed.

The object is further solved by an incineration plant through which combustion gases can flow and in which at least one measuring apparatus is positioned on an outer wall of the incineration plant where the coating probe can be inserted through an opening in the outer wall into an inner chamber through which combustions gases can flow.

It is advantageous if the coating probe can reach a region of the inner chamber in which at least one heat exchanger is provided.

A particular embodiment provides that the incineration plant comprises at least one cleaning device for the inner chamber as well as a monitoring unit, and the monitoring unit is adapted to cooperate with the at least one measuring apparatus and the at least one cleaning device.

Such an incineration plant can be operated by a method comprising at least the following steps:
a) determining a characteristic value for combustion residues in an inner chamber of the incineration plant by means of such a measuring apparatus,
b) evaluating the characteristic value in a monitoring unit with regard to a predefined limiting value,
c) performing at least one cleaning process in the inner chamber or an adaptation of the combustion process if the limiting value is reached.

In this method, when carrying out at least step a) or b) a temperature at a heat exchanger is taken into account.

An exemplary embodiment of a measuring apparatus is shown in the drawing and will be described in detail in the following. In the figures:

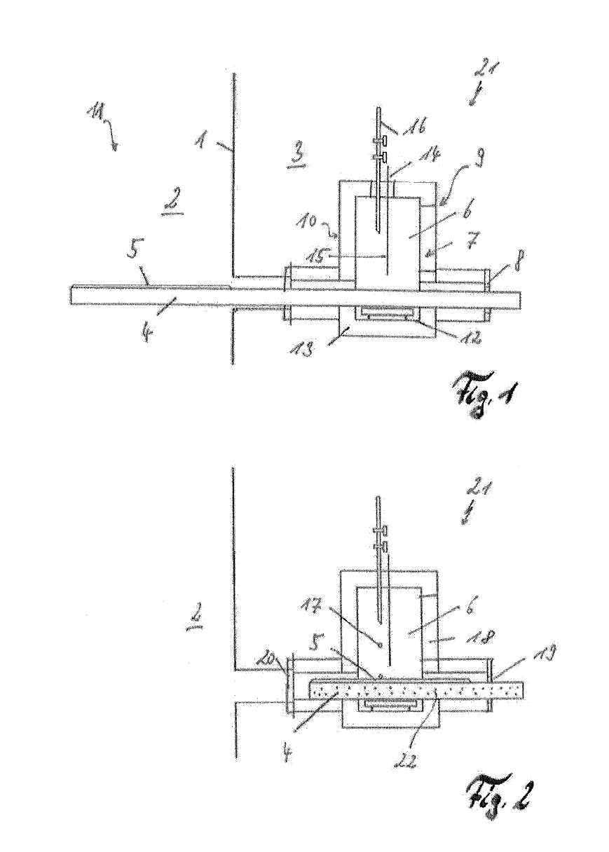

FIG. 1 shows schematically a measuring apparatus with coating probe pushed into a boiler chamber,

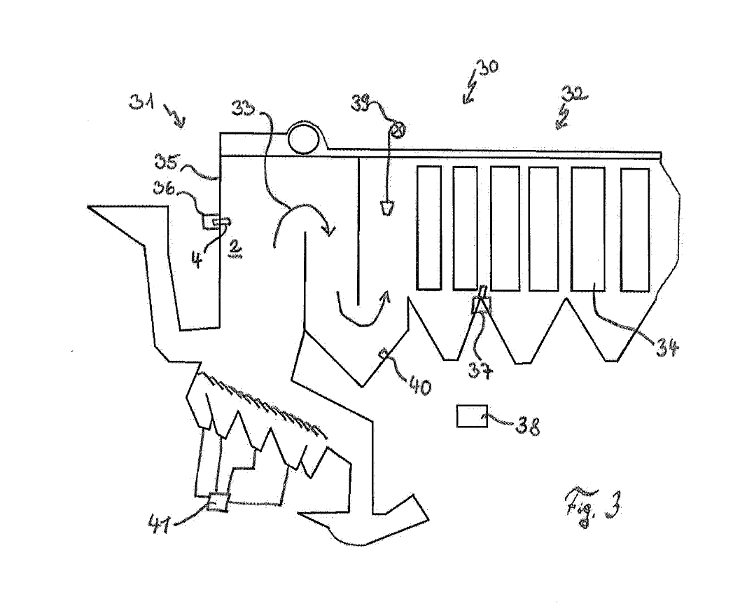

FIG. 2 shows schematically a measuring apparatus with coating probe drawn from the boiler chamber into a sample chamber and

FIG. 3 shows schematically a boiler system with measuring apparatus.

FIG. 1 shows the boiler wall 1 which separates a boiler chamber 2 as inner chamber from an outer chamber 3. A coating probe 4 is disposed in the boiler chamber 2 such that a coating layer 5 is deposited on the coating probe 4 due to flue gases flowing in the boiler chamber 2. Located in the outer chamber 3 is a sample chamber 6 through which the coating probe 4 is guided. A probe holder 8 located on the wall 7 of the sample chamber 6 allows the coating probe 4 to be pushed into the sample chamber on the side 9 of the sample chamber 6 and to be pushed out from the sample chamber 6 into the boiler chamber 2 on the opposite side 10.

This enables the coating probe 4 to be located in an incineration plant 11 and drawn directly from the incineration plant 11 into the sample chamber 6. FIG. 2 shows a withdrawn probe 4 which is located in the sample chamber 6.

The sample chamber 6 is heated by means of a hotplate 12 and thermally insulated by means of an insulating sleeve 13. A measuring window 14 enables a thermocouple 15 and a dropper 16 to be introduced into the sample chamber 6. The thermocouple 15 measures the temperature in the sample chamber 6 and the dropper 16 is arranged such that water drops 17 metered using this can be dropped onto the coating layer 5 of the coating probe 4. A viewing window 18 enables the processes in the sample chamber 6 to be viewed from outside.

A first sealable coating probe passage 19 seals the coating probe 4 towards the sample chamber 6 in the form of a gland seal. An opening 20 is provided opposite as coating probe passage which as a slider seals the sample chamber 6 against the boiler chamber 2. This enables the entire measuring apparatus 21 to be released from the incineration plant 11 without removing the coating probe 4 from the sample chamber 6.

In order that the coating probe 4 has a temperature at its surface which approximately corresponds to the heat transfer surfaces in the boiler chamber 2, fluid lines 22 for influencing the probe temperature are provided in the coating probe.

In practice, a heated gas or steam flow is initially passed through the coating probe with these fluid lines in order to heat the coating probe to a temperature of several hundred degrees Celsius. The coating probe 4 heated in this way is exposed to the flue gases in the boiler chamber 2 for several hours, days or weeks so that a coating layer 5 is deposited on the coating probe 4. The coating probe 4 with the coating layer 5 is then carefully transferred through the coating probe passage 20 into the sample chamber 6 so that the coating layer 5 on the coating probe 4 can be investigated in the sample chamber which is also heated to several hundred degrees Celsius.

After the investigations, the coating probe 4 or a new coating probe can be pushed through the sample chamber 6 into the boiler chamber 2.

Even if the temperatures in the sample chamber should be far lower than in the boiler chamber, the transfer of the probe from the boiler chamber directly into the sample chamber ensures that the coating does not absorb moisture from the atmosphere and does not vary substantially.

FIG. 3 shows an incineration plant 30 with a firing chamber 31 and a downstream so-called convection part 32 which both have combustion gases 33 flowing through them during operation. In the firing chamber 31 pipe coils (not shown) can be provided inside on the wall, forming a heat exchanger. In the area of the firing chamber 31 and/or the convection part 32, pipe coil packages suspended in the inner chamber can additionally (also) be provided as heat exchangers 34 which have combustion gases 33 flowing through and around them and form a heat exchanger.

In the exemplary embodiment a measuring apparatus 36 is mounted on the boiler wall 35 of the firing chamber 31 and on the convection part 32 a measuring apparatus 37 is mounted on the outer wall of the incineration plant 30. In the area of the measuring apparatus 36, 37, the outer wall has an (optionally closable) opening 20 through which a coating probe 4 can be temporarily inserted into the inner chamber. Here the coating probe 4 can preferably reach a region in the inner chamber in which a heat exchanger 34 is provided.

It is thus possible to generate a typical characteristic value for the coatings on a heat exchanger 34 by means of the measuring apparatus 36, 37. The determined characteristic value can then be evaluated by a monitoring unit 38. If it is found from this evaluation that a predefined limiting value and/or a limiting value adapted to the current operation of the incineration plant 30 (relating e.g. to the strength of the coating, the coating thickness, the type of coating, . . . etc.) is reached or exceeded, a cleaning process and/or an adaptation of the combustion process can be implemented.

The monitoring unit 38 is adapted to cooperate with the measuring apparatus 36, 37, the temperature sensor 40, the cleaning device 39 and/or adjusting means 41 for the incineration plant such as burner or air flaps. It is thus possible to clean the coatings on the heat exchangers 34 at a favourable time and/or influence the firing so that formation of coatings on the heat exchangers 34 is favourably influenced. In a supporting manner, a temperature at or in a heat exchanger 34 can be detected or determined so that, for example, the time for starting the step of evaluating the characteristic value or specifying the limiting value can be accomplished taking into account this temperature.

Water and/or steam dispensing devices mounted on the outer wall and/or which can be inserted through the outer wall, for example, come into consideration as cleaning device 39, in particular so-called water lance blowers, soot blowers, hose sprinklers et. In addition, beaters, (pressure) air cleaners and/or explosion generators can also be used.

1. Measuring apparatus comprising a coating probe (4) and a probe holder, wherein the probe holder (8) is disposed at a wall (7) of a sample chamber (6) and the coating probe (4) is displaceable on one side (9) into the sample chamber (6) and is displaceable on an opposite side (10) out from the sample chamber (6).

2. The measuring apparatus according to claim 1, wherein the sample chamber (6) is heated.

3. The measuring apparatus according to claim 1, wherein the sample chamber (6) is thermally insulated.

4. The measuring apparatus according to claim 1, wherein the sample chamber (6) has a measuring window (14).

5. The measuring apparatus according to claim 1, wherein the sample chamber (6) has a viewing window (18).

6. The measuring apparatus according to claim 1, wherein the sample chamber (6) has a dropper (16).

7. The measuring apparatus according to claim 1, wherein the sample chamber (6) has two opposite sealable coating probe passages (19, 20).

8. The measuring apparatus according to claim 1, wherein the coating probe (4) has fluid lines (22) for influencing the probe temperature.

9. The measuring apparatus according to claim 1, wherein the coating probe (4) comprises temperature measuring devices.

10. Method for investigating coatings on a coating probe (4) in which the coating probe (4) is disposed in an incineration plant (11), wherein the coating probe (4) is drawn from the incineration plant (11) directly into a sample chamber (6) heated to above 100° C. which adjoins the incineration plant (11) and is investigated there.

11. Incineration plant (30) through which combustion gases (33) can flow, wherein at least one measuring apparatus (36, 37) according to claim 1 is positioned on an outer wall (35) of the incineration plant (30) and the coating probe (4) can be inserted through an opening in the outer wall (35) into an inner chamber (2) through which combustions gases (33) can flow.

12. The incineration plant (30) according to claim 11, wherein the coating probe (4) can reach a region of the inner chamber (2) in which at least one heat exchanger (34) is provided.

13. The incineration plant (11) according to claim 11, wherein the incineration plant (30) comprises at least one cleaning device (39) for the inner chamber (2) as well as a monitoring unit (38), and the monitoring unit (38) is adapted to cooperate with the at least one measuring apparatus (36, 37) and the at least one cleaning device (39).

14. Method for operating an incineration plant (30) according to claim 11, comprising at least the following steps:
a) determining a characteristic value for combustion residues in an inner chamber (2) of the incineration plant (30) by means of a measuring apparatus (36, 37) comprising a coating probe (4) and a probe holder, wherein the probe holder (8) is disposed at a wall (7) of a sample chamber (6) and the coating probe (4) is displaceable on one side (9) into the sample chamber (6) and is displaceable on an opposite side (10) out from the sample chamber (6),
b) evaluating the characteristic value in a monitoring unit (38) with regard to a predefined limiting value,
c) performing at least one cleaning process in the inner chamber (2) or an adaptation of the combustion process if the limiting value is reached.

15. Method according to claim 14, wherein when carrying out at least step a) or b) a temperature at a heat exchanger (34) is taken into account.
